(19) United States
(12) Patent Application Publication
(10) Pub. No.: US 2023/0173278 A1
(43) Pub. Date: Jun. 8, 2023

(54) Methods and devices for treating itching

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 18/072,704

(22) Filed: Dec. 1, 2022

Related U.S. Application Data
(60) Provisional application No. 63/286,038, filed on Dec. 5, 2021.

(51) Int. Cl. A61N 1/36; A61N 1/04

(52) U.S. Cl. CPC A61N 1/36196; A61N 1/0408

(57) ABSTRACT

Methods and devices with noninvasive means for improving health such as treating itching especially skin itchiness are described. The non-invasive means is selected from electrical stimulation, heat or cold stimulation, IR (infrared) radiation, mechanical stimulation or their combinations. In some embodiments, the method and device utilizes electrical current passed through skin including the site having itchy feeling to achieve the desired therapeutical and beneficial effects.

### CROSS-REFERENCE TO RELATED APPLICATIONS

This application claims benefit of and priority to U.S. Provisional Patent Application Ser. No. 63/286,038 filed on Dec. 5, 2021, which is incorporated herein by reference in its entirety for all purposes. The entire disclosure of the prior application is considered to be part of the disclosure of the instant application and are hereby incorporated by reference.

### SUMMARY OF THE INVENTIONS

This application discloses methods and therapeutic devices with non-invasive means for improving health including treating itchiness, especially skin itchiness. The non-invasive means is selected from light stimulation, electrical stimulation, heat stimulation, cold stimulation, IR (infrared) radiation, mechanical stimulation (e.g. sound/vibrational stimulation) or their combinations. In some embodiments, the invention utilizes an electrical current passed through skin where the site has itchy feeling to achieve desired therapeutical and beneficial effects.

Preferably, the device in the invention is portable and self-contained, with a shape and means to be pressed against the skin area to be treated. It can be in a solid handheld shape/form such as that of a pen, a box, a cylinder, a cube, a stamp, a cone, or any other shape with a handle that can be held in the hand to place it against the skin so that one end of the device, with electrodes on the surface of that end, is in direct contact with or is near the skin. The device can produce electrical pulses or heat or cold or light (e.g. IR radiation) or vibration or sound, or their combination, and apply them to the said skin area.

In one aspect, the methods and devices described in the current invention use electro pulse or electrical pulses optionally in combination with vibration, heat or cold, or light such as IR radiation applied to the target skin areas to achieve the desired therapeutical effects. A person desiring to improve his or her health condition, such as by reducing itchiness, places the electrodes on the target skin areas and applies electrical stimulation through these electrodes. Heat and/or light and/or IR and/or vibration and/or cold stimulation produced by the device can also be applied to these areas for these purposes. The same device can also be used to improve brain function, such as by improving sleep or memory, treating senile dementia, or treating depression and insomnia, as well as by improving sleep, through means such as inducing deep sleep including slow wave sleep, which can also improve memory consolidation and improve cerebrospinal fluid flow which can clean metabolic products and toxic waste (e.g. amyloid) from the brain for better brain health. The stimulation pulse generating circuitry and power supply can be most conveniently packaged in a housing body structure with electrodes on its skin contact surface. One or more electrodes are on the skin contact surface of the housing which is placed in contact with skin. The device is placed so that the electrode/electrodes lies on the target skin area or on acupuncture points known to affect the targeted condition, e.g. the skin having an itchy feeling. Alternatively, the device is placed so that the electrodes are close to or overlie a nerve that runs under the acupuncture point, in which case the device may be placed some distance from the associated acupuncture point to provide a comfortable placement for the device.

The device can also contain one or more built-in heating elements that can keep the temperature of the skin contact surface between 40˜60° C. to provide a heating effect to the stimulation site/area. The device can also contain one or more built-in cooling elements that can keep the temperature of the skin contact surface between 0˜15° C. to provide a cooling effect to the stimulation site/area. The device can also contain one or more built-in visible light and/or IR radiation elements that can produce a light and direct it towards the skin contact surface to provide a visible light and/or IR stimulation effect to the stimulation site/area, as well as means to provide mechanical (e.g. vibrational) stimulation.

### BRIEF DESCRIPTION OF THE DRAWINGS

FIG. 1A shows an example of stimulation frequency in cycled pattern.

FIG. 1B shows examples amplitude modulated wave and frequency modulated wave.

FIG. 1C shows examples of pulse width modulation.

FIG. 2A shows an example of block diagram of the electrical circuitry suitable for the devices without additional heating elements.

FIG. 2B shows an example of block diagram of the electrical circuitry suitable for the devices with heating elements.

FIG. 3A shows an example of block diagram of the electrical circuitry suitable for the devices with electrodes, heating elements, speaker or vibrator.

FIG. 3B shows an example of the circuit diagram of pulse current generating circuit.

FIG. 4 shows examples of the device and its electrode assembly.

FIG. 5 shows examples of the skin contact surface of a device containing other type of device electrode assembly.

FIG. 6 shows examples of device electrode assembly with heating element.

FIG. 7 shows examples of device electrode assembly with light radiation element.

FIG. 8 shows examples of electrode assembly with IR radiation and heating functions.

FIG. 9 shows an exemplary device has a shape of a pen.

FIG. 10 shows another example of the device.

FIG. 11 shows an example of device that can also provide heating stimulation.

FIG. 12 shows an example of device having two skin contact surface containing electrodes.

FIG. 13 shows another example of a cone shape device that has two skin stimulation surface containing electrodes.

FIG. 14 shows example of a cube shape device that has two skin stimulation surface containing electrodes on the surface.

FIG. 15 shows example of device having detachable part.

FIG. 16 shows examples of the device having a status display.

### DETAILED DESCRIPTION

In some embodiments, the preferred (but not limited to) electrical pulse frequency used by the method and device of the current invention is between 2-1000 Hz or any combination of frequencies within 2-1000 Hz; the preferred pulse width is 0.001-50 ms and the preferred current is 0.5-100 milliamperes. One Hz means one pulse per second, despite the direction of the pulse.

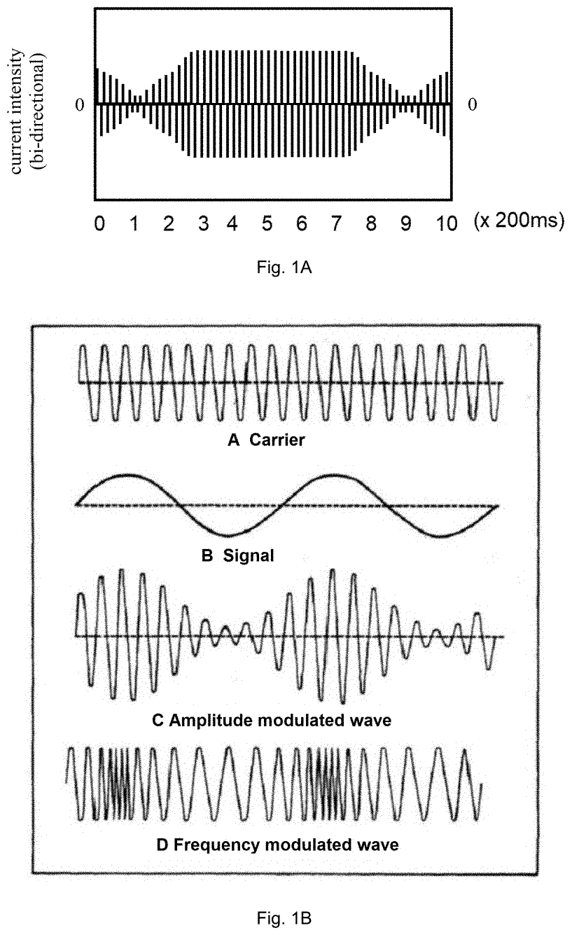

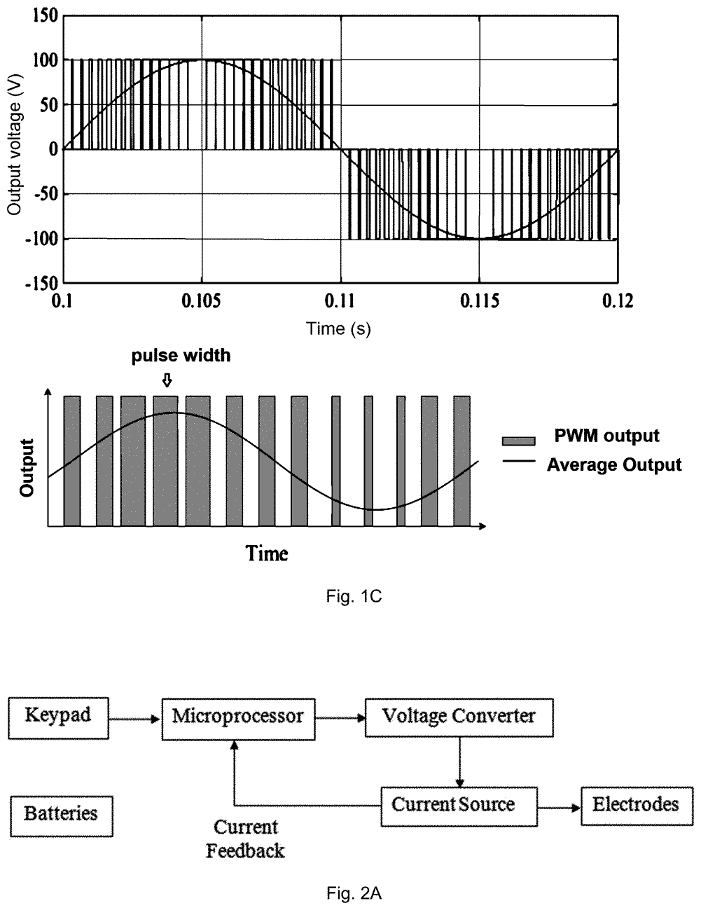

In some embodiments, the exemplary preferred electrical pulse repetition rate is approximately 2 pulses per second (2 Hz)with a pulse width of 600 microseconds, or approximately 15 pulses per second with a pulse width of 300 microseconds, or approximately 40 pulses (e.g. 30-50 or 35-45 pulses) per second with a pulse width of 200 microseconds, or approximately 100 pulses per second with a pulse width of 150 microseconds, or approximately 200 pulses per second (200 Hz) with a pulse width of 100 microseconds, or approximately 300 pulses per second (300 Hz) with a pulse width of 100 microseconds, or a higher frequency that the user feels most effectively reduces itching, or any combination of the above wave patterns that can provide effective results. The electrical current power levels can be about 0.1-50 mA at peak pulse height or any other power level that the subject in need feels comfortable and can effectively reduce itchiness. A wider range of pulse patterns can also be used in these non-invasive stimulation devices. Bi-directional electric pulse is preferred to avoid skin damage. The pulse and electric current flow change their directions during the electric pulse stimulations (e.g. as those shown in FIGS. 1A-1C). Here 1 Hz of stimulation means one pulse per second and the pulse can be of either direction. In general, a few seconds (e.g. 3-30 s) to a minute of stimulation on demand can provide effective treatment to the person in need of itchiness reduction. For many people, higher frequencies (Hz, also called pulse per second) or higher currents or wider peak width may provide better anti-itching effects, but may also produce an uncomfortable feeling upon stimulation; therefore, the user is given the option to adjust the frequency and current (which can be controlled by adjusting the voltage applied between electrodes) to achieve a good anti-itching effect while maintaining a comfortable feeling. In some preferred embodiments, the device has controls and electrical circuits that can provide variable output frequency, patterning and current intensity based on the user's input to achieve the best effect. In many embodiments, when square wave stimulation is used, it is desirable to have constant current output instead of constant voltage output, because the resistance of the skin changes when voltage being applied to the skin, which then may cause unpleasant feelings such as uncomfortable pain.

In some embodiments, the electrical pulse repetition rate of approximately 2 pulses per second with a pulse width of 600 microseconds, or approximately 15 pulses per second with a pulse width of 300 microseconds, or approximately 100 pulses per second with a pulse width of 200 microseconds, or the combination of the above wave patterns has been found to provide effective relief of itchiness in patients. The electrical power levels are about 2-50 milli-amps at peak pulse height. A wider range of pulse patterns can also be used in the non-invasive stimulation devices.

In one example, the stimulation is electrical pulse of alternating 2 Hz (0.6 ms pulse width) for 3 s and 15 Hz (0.3 ms pulse width) for 3 s at 1-5 mA. In another example, the stimulation is electrical pulse of alternating 2 Hz (0.6 ms pulse width) for 3 s and 100 Hz (0.2 ms pulse width) for 3 s at 1-3 mA. In another example, the stimulation is electrical pulse of alternating 2 Hz (0.6 ms pulse width) for 3 s, 15 Hz (0.3 ms pulse width) for 3 s and 100 Hz (0.2 ms pulse width) for 3 s at 1-3 mA. In another example, the stimulation is electrical pulse of alternating 50 Hz (0.3 ms pulse width) for 3 s and 100 Hz (0.2 ms pulse width) for 3 s at 1-3 mA. In another example, the stimulation is electrical pulse of alternating 100 Hz (0.1 ms pulse width) for 3 s and 100 Hz (0.2 ms pulse width) for 3 s at 1-3 mA.

In other examples, medium-frequency currents are used to treat itching. Typical medium frequency (e.g. 1 k-10 kHz) can stimulate the muscle and provide a massage like feeling to the area treated. In order to interrupt the current after each depolarization, rhythmic increases and decreases to the amplitude (amplitude modulation) can be applied. The amplitude modulation frequency (AMF) determines the frequency of the depolarization. The AMF corresponds to the frequencies used in low-frequency electrotherapy. The amplitude modulation can also be replaced by pulse width modulation.

The electric current pattern used in modulated medium frequency electrotherapy can be used in the devices of the current invention. Suitable frequency can be 2 k-10 kHz. The combination of low frequencies and medium frequencies can be used, e.g. the combination of frequencies of 1-250 Hz low frequency current and 2 k-10 kHz medium frequency current. The low and/or medium frequency can have a variety of wave shapes/patterns (e.g. sinusoidal wave, square wave, continued, discontinued, varying amplitude, symmetrical wave or unsymmetrical wave etc.). The amplitude of medium frequency can vary at low frequency patterns. In some embodiments, suitable current is between 0.05-5 mA.

A gamma wave is a pattern of neural oscillation in humans with a frequency between 25 and 100 Hz, though 40 Hz is typical. It can benefit and improve brain function. Light, sound, vibration, electric frequency, electromagnetic or magnetic stimulation that can stimulate gamma waves in brain can be used for the current invention, for example those between 25-100 Hz, preferably between 30-80 Hz. Stimulation having a frequency can induce the brain to resonate in a similar frequency. In some embodiments, the stimulation frequency, such as the electrical or light or sound pulse or other mechanical (e.g. vibrational) frequency is between 30-60 Hz. In some embodiments, the stimulation frequency such as the electrical pulse frequency is between 35-50 Hz. In some embodiments, the stimulation frequency such as electrical pulse frequency is 40 Hz. In some embodiments, the frequency is 40 Hz and the peak width is between 0.1-0.6 ms, e.g. 0.3 ms for electrical pulses. The peak width for sound, light and other mechanical stimulations can be different, e.g. 1 ms-10 ms.

In some preferred embodiments, the stimulation frequency such as electrical pulse is applied in cycled batch; in each cycled batch, the current goes from low to high and then keeps constant for a certain amount of time, such as 1-10 s, then drops back to low gradually for the next cycled batch. For example, as shown in FIG. 1A, a ˜40 Hz stimulation electric pulse starts from a low to high current in ˜300 ms and then keeps the current constant for ˜1000 ms, then drops back to a low current gradually in ˜300 ms until the next cycled batch. There can be an interval period (i.e. with no stimulation pulse) between each cycled batch, e.g. one lasting 1-10 seconds. The combination of different frequencies of gamma waves or other frequency ranges can also be used; for example, the frequency can first be 35 Hz for 5 seconds, then 40 Hz for 5 seconds, then 45 Hz for 5 seconds, and back to 40 Hz for 10 seconds. Similarly, stimulation other than electric pulse, such as light, temperature, and vibration, can also be applied in cycled batch.

In some embodiments, medium-frequency stimulation such as electrical current or light or vibration are used to give low frequency stimulation (e.g. 1 Hz˜300 Hz stimulation). A typical medium frequency (e.g. 1 k-10 kHz as carrier wave frequency) can be used. In order to give low frequency wave stimulation, rhythmic increases and decreases of the amplitude (amplitude modulation) can be applied at low frequency wave frequency. The amplitude modulation frequency (AMF) determines the frequency of the low frequency wave signal. The AMF corresponds to the frequencies used in low-frequency signal stimulation used in therapy. Therefore, the stimulation such as electric current pattern used in modulated medium frequency electrotherapy can also be used in the devices of the current invention. Suitable carrier frequency can be 2 k-10 kHz. For example, the amplitude of medium frequencies can vary at low frequency patterns at the frequency of gamma wave to generate gamma wave stimulation. The combination of low frequency and medium frequency can be used, for example a combination of low frequency i.e. 30-200 Hz frequency stimulation such as electric current stimulation and 2 k-10 kHz medium frequency such as electric current stimulation. The low and/or medium frequencies can have varieties of wave shapes/patterns (e.g. sinusoidal wave, square wave, continued, discontinued, varying amplitude, symmetrical wave or unsymmetrical wave etc.). In some examples, the suitable current is between 0.05 and 5 mA. If other stimulation is used such as light or sound, the intensity used should be tolerable by the user or be comfortable to the user. FIG. 1B shows exemplary pattern of amplitude modulated wave and frequency modulated wave.

Similarly, a light stimulation can also be given to a subject in need for the same indication. The light stimulation can use medium or high frequency light to give low frequency stimulation. Typical medium frequency (e.g. 1 k-10 kHz or higher as carrier frequency) can be used. In order to give low frequency wave stimulation, rhythmic increases and decreases to the amplitude of light intensity (amplitude modulation) can be applied at gamma wave frequency. The amplitude modulation frequency (AMF) determines the frequency of the gamma wave. The AMF corresponds to the frequencies used in low-frequency light stimulation. Therefore, the light pattern in modulated medium frequency flash (flicker) can be used in the devices of the current invention. Suitable carrier frequency can be 2 k-10 kHz or higher frequency such as 10 k-1 MHz. In some embodiments, the carrier frequency is between 2 k-10 kHz. For example, the amplitude of medium frequencies can vary at low frequency patterns at the frequency of gamma waves to generate gamma wave stimulation. The combination of low frequency and medium frequency can be used, e.g. the combination of low frequency i.e. 10-300 Hz (such as 40 Hz gamma waves) light flicker and 2 k-10 kHz medium frequency light flicker. The low and/or medium frequency can have variety of wave shapes/patterns (e.g. sinusoidal wave, square wave, continued, discontinued, varying amplitude, symmetrical wave or unsymmetrical wave etc.). The amplitude of medium frequency can vary at the low frequency pattern. The simulation intensity and duration of stimulation used should be tolerable by the user or be comfortable to the user.

Similarly, a vibration or other mechanical stimulation can also be given to a subject in need for the same indications. The vibration or other mechanical stimulation can use medium or high frequency sound/vibration to give low frequency stimulation. Typical medium frequency (e.g. 1 k-20 kHz) can be used. In order to give low frequency stimulation, rhythmically increasing and decreasing the amplitude of sound/vibration or other mechanical stimulation intensity (amplitude modulation) can be done at low frequency. The amplitude modulation frequency (AMF) determines the frequency of low frequency stimulation. The AMF corresponds to the frequencies used in low-frequency vibration stimulation. Therefore, the vibration or other mechanical stimulation pattern in modulated medium frequency can be used in the devices of the current invention. A suitable frequency can be 2 k-10 kHz or a higher frequency such as 10 k-100 kHz. Lower frequencies such as 100 Hz-1000 Hz can also be used. In some embodiments, suitable carrier frequencies can be 0.2 k-10 kHz. The amplitude of medium frequency can vary at the low frequency pattern at the frequency of low frequency wave to generate low frequency stimulation. The combination of low frequency and medium frequency can be used, e.g. the combination of low frequencies of 30-100 Hz (such as 40 Hz) gamma waves sound/vibration/other mechanical stimulation and 2 k-10 kHz medium frequency sound/vibration/other mechanical stimulation. The low and/or medium frequency can have a variety of wave shapes/patterns (e.g. sinusoidal wave, square wave, continued, discontinued, varying amplitude, symmetrical wave or unsymmetrical wave etc.). The amplitude of the medium frequency can vary in low frequency patterns. The intensity and duration of stimulation used should be tolerable by the user or be comfortable to the user.

In some embodiments, the vibrational stimulation used to reduce itchiness is ultrasound stimulation, e.g. those at frequencies between 20 kHz˜100 kHz. The frequency can also be in a higher range such as up to 200 MHz.

Alternatively, frequency modulated wave (FMF) or PWM (pulse width modulation) stimulation can be used instead of or in combination with amplitude modulation frequency (AMF) to achieve the desired low frequency stimulation (e.g. ˜40 Hz frequency) using medium carrier frequency (e.g. 1 k-10 kHz) or high frequency stimulation for light or electric or sound/vibration/other mechanical stimulation or visible light or IR stimulation. PWM (pulse width modulation) is widely used in OLED display. A similar technique can be readily adopted for the current invention. FIG. 1C shows several formats of pulse width modulations. By varying the pulse width at the switching frequency in PWM, stimulation at the desired frequency (e.g. ˜40 Hz gamma frequency or delta wave frequency) can be achieved. For example, suitable PWM switching frequencies can be 1 k-10 kHz or higher such as 10 k-1M kHz. In some embodiments, Suitable PWM switching frequency can be 10 k-100 kHz. The low and/or high frequency can have a variety of wave shapes/patterns (e.g. sinusoidal wave, square wave, continued, discontinued, varying amplitude, symmetrical wave or unsymmetrical wave etc.). The amplitude of the PWM frequency can vary at low frequency patterns. The intensity and duration of stimulation used should be tolerable to the user or be comfortable to the user.

In some embodiments, the device and method of the current invention use alpha (7.5-14 Hz) or theta (4-7.5 Hz) or delta (around 0.1-4 Hz) wave frequencies/pulse stimulation or their combination instead of using gamma wave stimulation. The stimulation can be selected from the following: electric pulse, electromagnetic wave, magnetic field, visible light, IR, sound/vibration/other mechanical stimulation, or their combinations. Stimulation with a certain frequency can induce the brain to resonate at a similar frequency. The stimulation can have the frequency at alpha (7.5-14 Hz) or theta (4-7.5 Hz) or delta (0.5-4 Hz) or zeta (0.1-0.5 Hz). The format and generation of these alpha (7.5-14 Hz) or theta (4-7.5 Hz) or delta (0.5-4 Hz) or zeta (0.1-0.5 Hz) stimulation waves can be similar to those in gamma wave stimulation as described previously, e.g. they can be directly at the frequency of alpha (7.5-14 Hz) or theta (4-7.5 Hz) or delta (0.5-4 Hz) or zeta (0.1-0.5 Hz) waves or indirectly using amplitude modulation frequency (AMF) or FMW (frequency modulated wave) or PWM (pulse width modulation) or their combination, similar to those in gamma frequency as described previously. Although delta wave frequency is described as 0.5-4 Hz frequency in some literatures, zeta (0.1-0.5 Hz) wave frequency is considered as a subset of delta wave frequency. The difference of frequencies between different brain waves is not distinct and there is no clear definition. For example, some publications define delta wave frequency as those <3 Hz instead of 0.5-4 Hz or 0.1-4 Hz. In the current inventions, delta wave frequency is defined as a wave having a frequency between 0.1-4 Hz.

In embodiments of the current inventions, the stimulation frequency such as electrical or light or sound/vibration pulse frequency or other mechanical stimulation (for example, applying mechanical force/pressure periodically at certain frequency is considered vibration) is between 0.1-4 Hz as a delta frequency. The term mechanical stimulation in the current inventions include sound/vibration stimulation. Sound stimulation is essentially a vibration stimulation. The term vibration in the current inventions include mechanical stimulation such as applying mechanical force/pressure to target area periodically at certain frequency (e.g. pulse). The term vibrator means a physical mean that can generate a vibration to a target area. Examples of vibrators can be found in the device to provide a massaging effect, such as those used in commercial massagers. For example, the vibrator can be a device that provides a kneading effect such as shiatsu. Rotating wheels or inflatable airbags can be used to apply pressure to body parts to provide a massage effect. The inflatable airbags can inflate and deflate periodically to provide cycled pressure as a massaging effect. In some embodiments, the delta wave stimulation frequency, such as electric pulse frequency, light pulse frequency, or vibration frequency, is between 1-3 Hz. In some embodiments, the stimulation frequency, such as electric pulse frequency, light pulse frequency, or vibration frequency, is 2 Hz. In some embodiments, the stimulation frequency, such as electrical pulse frequency, light pulse frequency, or vibration frequency, varies between 0.5-4 Hz repeatedly. In some embodiments, the electrical pulse frequency is 0.3-2 Hz and the peak width is between 0.1-10 ms, e.g. 1 ms. In some embodiments, the light or vibration pulse frequency is 1-2 Hz and the peak width is between 10-400 ms, e.g. 200 ms. In some embodiments, the light or vibration pulse frequency is 0.5-1 Hz and the peak width is between 100-1000 ms, e.g. 500 ms. In some embodiments, the stimulation, such as electrical pulse, light pulse, or vibration, is applied in cycled batch, and in each cycled batch the current goes from low to high and then keeps constant for a certain amount of time such as 5-10 s, then drops to low gradually for the next cycled batch as shown in the FIG. 1a. There can be an interval period (no electric or light or vibration pulse) between each cycled batch, e.g. 1-10seconds. The combination of different frequency of delta wave can also be used, for example the frequency is 3 Hz in the first 15 s (seconds) and then 2 Hz for 15 s and then 1 Hz for 15 s and then and then 0.5 Hz for 15 s goes back to 1 Hz for 15 s. The combination of different brain wave stimulation forms can also be used. In some embodiments, the stimulation is applied at the order of reduced frequency, for example optional gamma (35-45 Hz) wave stimulation for 15 s and then alpha (7.5-14 Hz) wave stimulation for 15 s and then theta (4-7.5 Hz) wave stimulation for 10 s and then delta wave stimulation for 30 s.

In some embodiments, medium-frequency stimulation such as electrical current or light or sound or vibration are used to generate delta wave stimulation. A typical medium frequency (e.g. 1 k-10 kHz as carrier wave frequency) is used. In order to give delta wave stimulation, rhythmically increasing and decreasing the amplitudes (amplitude modulation) can be applied at delta wave frequency. The amplitude modulation frequency (AMF) determines the frequency of the delta wave signal. The AMF corresponds to the frequencies used in low-frequency signal stimulation used in therapy. Therefore, the stimulation such as electric current pattern used in modulated medium frequency electrotherapy can also be used in the devices of the current invention. Suitable carrier frequency can be 1 k-50 kHz. The amplitude of medium frequency can vary at the low frequency pattern at the frequency of delta wave to generate delta wave stimulation. The combination of low frequency and medium frequency can be used, e.g. the combination of low frequency of 0.1-4 Hz delta wave frequency stimulation such as electric current and 2 k-10 kHz medium frequency such as electric current. The low and/or medium frequency can have a variety of wave shapes/patterns (e.g. sinusoidal wave, square wave, continued, discontinued, varying amplitude, symmetrical wave or unsymmetrical wave etc.). In some examples, a suitable current is between 0.05-5 mA. If other stimulation is used such as light or sound, the intensity used should be tolerable by the user or be comfortable to the user.

Similarly, a delta wave light stimulation can also be given to a subject in need for the same indications. The light stimulation uses medium or high frequency light to give delta wave stimulation. Typical medium frequency (e.g. 1 k-10 kHz or higher as carrier frequency) can be used. In order to give delta wave stimulation, rhythmically increasing and decreasing the amplitude of light intensity (amplitude modulation) can be applied at delta wave frequency. The amplitude modulation frequency (AMF) determines the frequency of delta wave. The AMF corresponds to the frequencies used in low-frequency light stimulation. Therefore, the light pattern in modulated medium frequency flash (flicker) can be used in the devices of the current invention. Suitable carrier frequency can be 2 k-10 kHz or higher frequency such as 10 k-1 MHz. In some embodiments, a suitable carrier frequency can be 2 k-10 kHz. The amplitude of medium frequency can vary at the low frequency pattern at the frequency of delta wave to generate delta wave stimulation. The low and/or medium frequency can have a variety of wave shapes/patterns (e.g. sinusoidal wave, square wave, continued, discontinued, varying amplitude, symmetrical wave or unsymmetrical wave etc.). The amplitude of medium frequency can vary at the low frequency pattern. The simulation intensity and duration of stimulation used should be tolerable by the user or be comfortable to the user.

Similarly, a sound and/or vibration (sound/vibration) stimulation can also be given to a subject in need for the same indications. The sound/vibration stimulation can use low or medium or high frequency sound/vibration to give delta wave stimulation. Typical low frequency (e.g. 20 Hz-1 K Hz) or medium frequency (e.g. 1 k-20 kHz) can be used. In order to give delta wave stimulation, rhythmically increasing and decreasing the amplitude of sound/vibration intensity (amplitude modulation) can be applied at delta wave frequency. The amplitude modulation frequency (AMF) determines the frequency of delta wave. The AMF corresponds to the target frequencies used in sound/vibration stimulation. Therefore, the sound/vibration pattern used in modulated low/medium frequency can be used in the devices of the current invention. Suitable carrier frequencies can be 2 k-10 kHz or higher frequency such as 10 k-100 kHz. Lower carrier frequency such as 20 Hz-1 kHz can also be used. In some embodiments, a suitable carrier frequency can be 20 Hz-10 kHz. In some embodiments, a suitable carrier frequency can be 100 Hz-1 kHz. The amplitude of carrier frequency can vary at the low frequency pattern at the frequency of delta wave to generate delta wave stimulation. The combination of low frequency and medium frequency can be used, e.g. the combination of low frequency of 0.1-5 Hz (such as 2 Hz) delta wave frequency sound/vibration and 2 k-10 kHz medium frequency sound/vibration. The low and/or medium frequency can have a variety of wave shapes/patterns (e.g. sinusoidal wave, square wave, continued, discontinued, varying amplitude, symmetrical wave or unsymmetrical wave etc.). The amplitude of medium frequency can vary at the low frequency pattern. The intensity and duration of stimulation used should be tolerable by the user or be comfortable to the user.

Alternatively, PWM (pulse width modulation) or FMW stimulation can also be used instead of or in combination with amplitude modulation frequency (AMF) to achieve the desired low frequency stimulation (e.g. ˜2 Hz) using low/medium frequency (e.g. 1 Hz˜10 kHz) or high frequency stimulation for light or electric or sound/vibration or visible light or IR stimulation. PWM (pulse width modulation) is widely used in OLED display. The similar technique can be readily adopted for the current invention. By varying the pulse width at the carrier frequency (e.g. ˜2 k Hz frequency), stimulation at desired frequency (e.g. ˜2 Hz delta frequency) can be achieved. For example, suitable PWM switching frequency can be 1 k-10 kHz or higher frequency such as 10 k-1M kHz. In some embodiments, Suitable PWM switching frequency can be 10 k-100 kHz. The low and/or high frequency can have a variety of wave shapes/patterns (e.g. sinusoidal wave, square wave, continued, discontinued, varying amplitude, symmetrical wave or unsymmetrical wave etc.). The amplitude of PWM frequency can vary at low frequency patterns. The intensity and duration of stimulation used should be tolerable by the user or be comfortable to the user.

The devices described in the current invention can generate gamma, delta, alpha or theta frequency stimulations to a subject to improve brain function such as improving sleep quality and memory, and treating senile dementia including Alzheimer's disease. They can also reduce itching when the stimulation is applied to the skin area having itchiness or the skin area close to the area having itchiness.

The current invention discloses device having means to provide electric pulse or electromagnetic waves or magnetic fields or visible light or IR or sound/vibration or heating or cooling or their combinations to improve body functions such as anti-itching. The device contains means to deliver said stimulation at a preset frequency. In some embodiments, the device contains means to produce electric pulse. The device can also contain one or more built in heating means that can keep the temperature of the skin contact surface between 35˜60 degrees Celsius to provide a heating effect to the stimulation sites/area. The heating means can be an electrical heating element powered by a battery. The preferred heating temperature at the surface is between 37˜55 degrees Celsius. In some embodiments the heating temperature at the surface is between 40˜50 degrees Celsius. The device can have a sensor to detect the temperature at its skin contact surface to turn on or off the heater to maintain the temperature at target level. An exemplary heating element is self-regulating heater such as polymer PTC heating elements or ceramic PTC heater or carbon fiber/sheet heater. MTC (Metal Ceramics Heater) can also be used. Resistive heaters can be made of MTC or conducting PTC rubber materials or ceramic PTC material where the resistivity increases exponentially with increasing temperature. Such a heater will produce high power when it is cold, and rapidly heat up itself to a constant temperature. Due to the exponentially increasing resistivity, the heater can never heat itself to warmer than this set temperature. The temperature can be chosen during the production of the rubber or ceramic. The heating step can be cycled to avoid skin desensitization. For example, each heating step is 0.5-3 s followed by 0.5-3 s none heating period.

The device can also contain one or more built in visible light and/or IR radiation element that can produce visible light and/or IR radiation for either thermal or non-thermal effect and pass it to the skin contact surface or area of interest (e.g. to provide a photon stimulation and/or heating effect to the stimulation sites/area. The visible light or IR radiation elements can be tungsten wire, carbon, or alloys of iron, chromium, and aluminum as well as LED and laser or their combinations to provide broader wavelength coverage. It can also use laser that has narrow bandwidth. IR radiation elements are widely used in physical therapy and they can be adapted readily for the current invention. The device can have one centralized visible light and/or IR radiation elements connected to multiple optical fibers, which can transfer the visible light and/or IR radiation to the desired stimulation sites. The surface of the housing body can have one or more light transparent windows to allow the photon emitted from the light source reaching the skin. Suitable wavelength of IR can be between 700 nmm-1 mm. It can be either NIR (near IR) or MIR or FIR or its combination. In some embodiments, it is between 800 nm-100 um. In other examples, it is between 1 um-20 um. The output power of IR radiation can be adjusted to provide effective and safe radiation. When heating effect is desired, preferably the suitable power level can heat the target skin site to 40° C.-50° C. but does not burn/damage the skin and is acceptable by the user. The radiation can be either continuous or pulsed as those used in the electric pulse stimulation described previously. The intensity and duration of stimulation used should be tolerable by the user or be comfortable to the user.

In one example, a 10 W IR radiation element with 2 μm-10 μm wavelength output is used; the output is coupled to IR optical fibers to deliver IR radiation to the treatment sites. In another example, a 808 nm GaAlAs laser or 1550 nm laser can be used, connected to multiple optical fibers to deliver IR pulse to the sites and the gamma wave frequency between 30-50 Hz at the power level of 10-1000 mW/cm² can be applied. When IR LED is used, each stimulation site can have its own IR LED attached without the need of using optical fibers. Red light and NIR radiation can penetrate into deep tissue and improve mitochondrial activity and activate cell energy metabolism.

In some embodiments, the current invention discloses a method for reducing itching in a subject in need thereof, the method comprising administering a non-invasive electrical stimulus have one or more frequency of about 5 Hz to about 500 Hz to the subject's itching skin surface area. Additional heating and/or cooling and/or NIR and vibrational stimulation can also be applied to the same area. The current invention also discloses a device that have means to generate a non-invasive electrical stimulus having a frequency of about 5 Hz to about 500 Hz to be applied to the subject's itching skin surface area. The device contains an electrical stimulus generating source and has a shape that can be hold with hand to press it against skin.

The method for improving skin health and treating itchiness can also be combination of electrical stimulus, heat, cold, sound/vibration and visible light/IR (non-heating effect) described previously. In general, higher and longer stimulation level can provide better effect but the intensity and duration of stimulation used should be tolerable by the user or be comfortable to the user and should not cause damage to the treated area/tissue and the subject. The user can adjust the device output intensity to achieve the desired level. Different type of stimulation can be applied simultaneously or sequentially. For example, electric stimulation and light stimulation can be applied to a subject at the same time concurrently, or they can be applied to a subject alternately or sequentially.

To improve skin health and cell activity and treating itchiness, the wavelength of the light to irradiate skin area (none-eye area) can be between 500-1500 nm, or 550-1100 nm such as those having peak wavelength at 590 nm or 670 nm or 780 nm or 790 nm or 808 nm or 810 nm or 850 nm or 980 nm or 1064 nm or their combinations. In some embodiments it is selected from red light/NIR (near IR) such as those with peak at 633, 670, 810, 850, 980 and 1064 nm. In some embodiments it has a peak wavelength at 808-820 nm. In some embodiments it has a peak wavelength at 633 nm or 670 nm or 810 nm or 850 nm. In some embodiments the light intensity can be 10-1000 mW/cm². For example, they can be multiple light emitting units such as LEDs having 0.5-6 W power level each with total power of 5-200 W, and the light output of each light emitting unit (e.g. LED) can be 50-600 lumens each. In some embodiments the irradiation can be on demand basis or 1 min-100 min 1-3 times a day for 1-10 weeks or until desired effect is achieved. The intensity of the light needs to be not too high that can cause damage to the skin of the irradiation area. The light stimulation can be in the frequency pattern described previously or non-pulsed (continuous).

Optionally, the device of the current invention can have a communication module that can communicate with an external control (command) module to receive commands of the stimulation output (e.g. stimulation type, time, frequency, on/off, power level and pulse pattern) and produce stimulation accordingly. The external control module can be a remote or computer or a cell phone with dedicated application installed. The communication can utilize Wi-Fi or bluetooth or IR or radio signal. The device can have an on/off control to turn on or turn off the communication module.

The electrical circuitry of the device can be implemented with well-known art. There are many designs to implement the circuitry. FIG. 2A illustrates an example of the circuit diagram suitable for the device. The microprocessor receives input from the keypad or other input means on the device to set the intensity level of the stimulation such as electric pulses, and current feedback to regulate the microprocessor output to voltage converter and current source to generate the stimulate pulses at the current level set by the keypad. The voltage converter converts battery voltage to a target voltage to drive the stimulation output. The output of the current source is connected to the electrodes, which contact skin. The batteries such as a rechargeable battery pack supply power to every block.

Similarly, visible light/IR and/or sound/vibration and/or heating and/or cooling stimulation generating circuit can also be incorporated into the device and controlled by a microprocessor, which is further controlled by keypad or other input means on the device or remote control means such as cell phone app. The stimulation end (e.g. electrode, vibrator, LED) can be built within the main device body with other components or separate with the main body containing other component and connected to the main body with wire and/or optical fibers to provide better portability and user convenience. In some preferred embodiments, the stimulation end that contacts skin (e.g. electrode, vibrator, heat stimulation surface) is on the surface of the main device body and not separated from the main body.

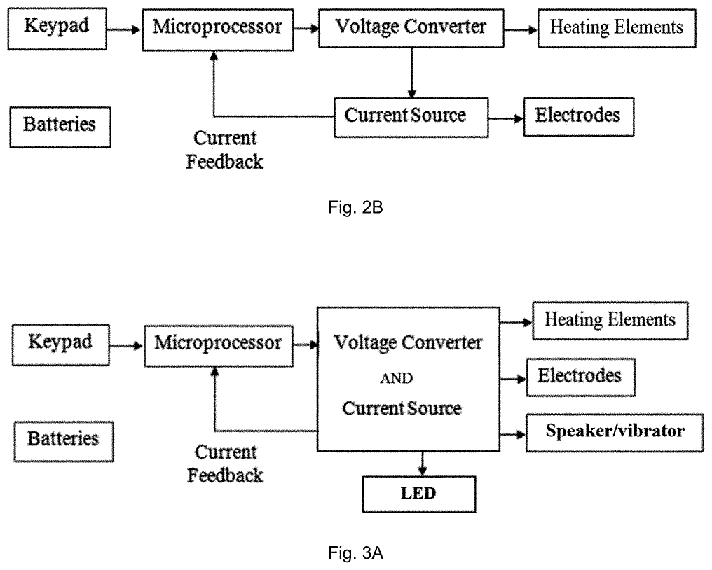

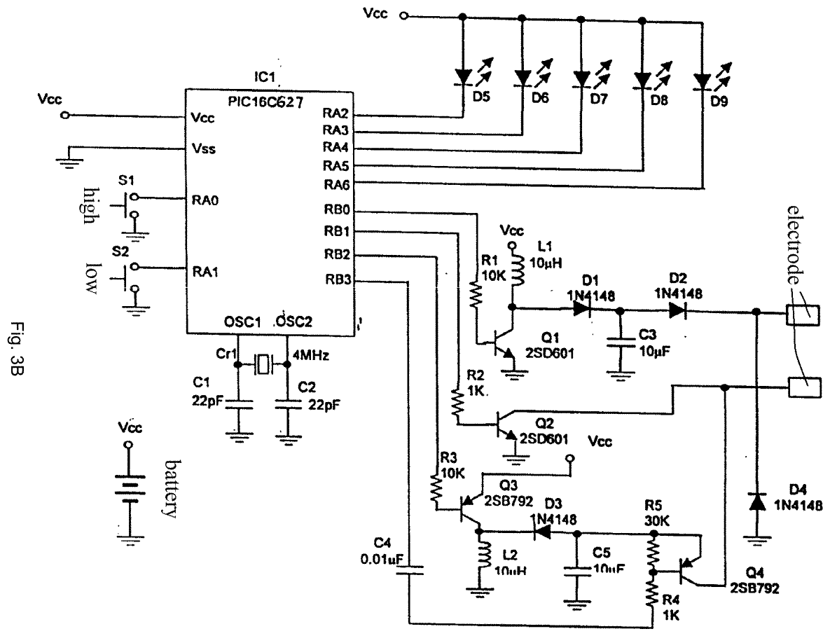

FIG. 2B illustrates an example of the circuit diagram suitable for the device with additional heating elements controlled by microprocessor and voltage converter. The voltage converter provides two voltages to drive the heating elements and electrodes. FIG. 3A illustrates an example of the circuit diagram suitable for the device with electrodes, heating elements, light emitting LEDs and sound/vibration generating speaker/vibrator controlled by microprocessor.

The electrical circuitry of the device can be implemented with well-known art. There are many designs to implement the circuitry. In examples the microprocessor receives input from the keypad to set the intensity level of the stimulate pulses, and current feedback to regulate the microprocessor output to voltage converter and current source to generate the stimulate pulses at the current level set by the keypad. The voltage converter converts battery voltage to high voltage. The output of the current source is connected to the electrodes, which contact skin. The batteries supply power to every block. FIG. 3B illustrates an example of the circuit diagram of pulse current generating circuit, which uses a programable microprocessor IC1 as master controller. IC1 accept commands from current intensity control button S1, S2 to controls the on/off of light emitting diode D5-D9 indicator which shows the stimulation level. Stimulation pulse is generated by the boost circuit consisting of triode Q1, Q3, inductance L1, L2, capacitor C3, C5 and diode D1, D3; and stimulation control circuit consisting of triode Q2, Q4, diode D2, D4. IC1 is switching on and off triode Q1, Q3 to enable inductance L1, L2 to charge capacitor C3, C5 through diode D1 and D3. The charging voltage is determined by the on/off switch number of Q1/Q3 controlled by IC1 based on the command from S1 and S2. Therefore the stimulating current is controlled. When the voltage of capacitor C3, C5 reaches predetermined value, microprocessor IC1 connects triode Q2, Q4 to allow the voltage from capacitor C3, C5 being applied on the electrode, produces stimulating current. The pulse frequency range is 5˜150 hertz, and peak width is 1˜500 microsecond, and current intensity is 1˜100 mA. The wave amplitude modulation is realized by microprocessor IC1 programming.

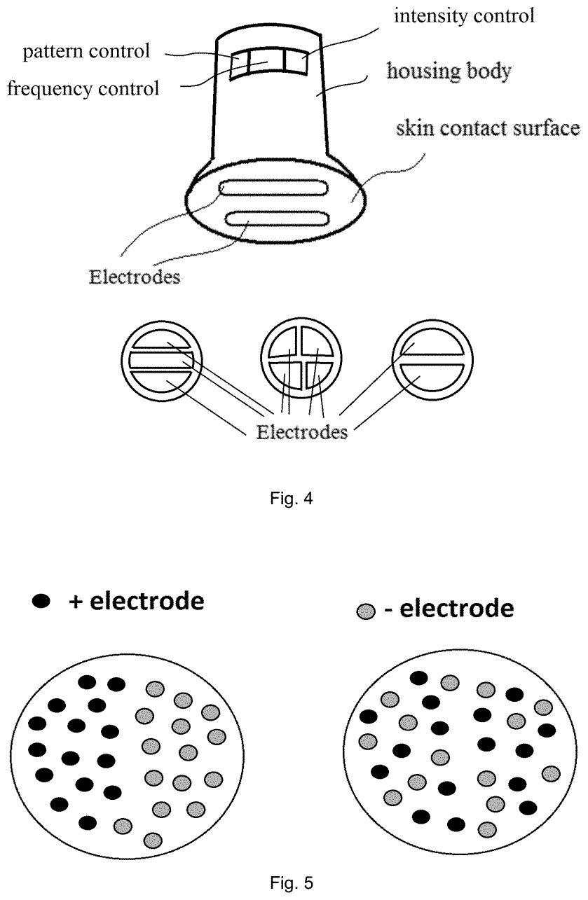

FIG. 4 shows one embodiment of the device and its electrode assembly. In FIG. 4 top on the skin-contacting surface of the device body, two electrodes are aligned in a side by side format. One electrode functions as positive (or connects to the hot wire of the current source) and another electrode functions as negative (or connects to the neutral wire). It also has 3 control buttons on its housing body, to control the current intensity (e.g. from no current/off to highest current such as 10 mA), pulse frequency (e.g. 5˜500 Hz) and stimulation pattern (e.g. continuous, intermittent or cycling between several different frequency). The electrode can be made of metal or gel pad such as those used in TENS device or other electricity conducting material. One electrode functions as positive and another electrode functions as negative and they can change polarity during stimulation since the pulse and current flow are bi-directional. Different shape of electrode and more than two electrodes can be placed on the skin contact surface as shown in FIG. 4 bottom. Bottom left shows the skin contact surface has 3 electrodes. Bottom middle shows the skin contact surface has 4 electrodes. Bottom right shows the skin contact surface has 2 semicircle shape electrodes. Some electrodes serve as positive and other electrodes serve as negative to form the closed loop for current once they are attached to the skin and they change polarity during stimulation since the pulse and current flow are bi-directional (e.g. as shown FIGS. 1A-1C).

The term electrode assembly means a structure that has one skin contact surface and at least 2 electrodes placed on this skin contacting surface. Electrode assembly is the component of the device that in contact with target skin area to be stimulated. When the electrodes are attached to the skin, the electrical pulse is applied to an individual's skin via two or more electrodes, and the current is conducted between electrodes through soft tissue and skin.

As shown in FIG. 4, the control, power supply and stimulation pulse generating circuitry for the device can be integrated within a handhold housing body. The housing can also be in other shape and format as long as it can be held with hand readily. The intensity (power level) control buttons control the output power level of the electrodes. Higher power level (the intensity of the stimulation current) generally gives stronger stimulation, which may generate better therapeutic effects. The person in need uses the control buttons to adjust the power level to achieve desired therapeutic effects and the best comfort. Other buttons may also be used to control the pulse patterns and frequency. An optional LCD display displays the working status of the device such as the current power level and pulse pattern. A timing function can also be incorporated within the LCD. The housing sends out electrical pulse to the electrode assembly to apply electrical stimulation and/or other stimulation (e.g. light, sound stimulation) to the person in need.

The device in FIG. 4 can also contains means to provide heating, visible light/IR radiation, mechanical stimulation to skin as previously described. The heating/light/IR stimulation can be applied at the same time with electric stimulation or be applied alternately or sequentially. The device can contain a sound/vibration generating means such as one or more speaker/vibrator, which can be attached to skin. As previously described, the means to generate light/sound/vibration (e.g. LED, speaker) can be integrated in the electrode assembly. In some embodiments the device contains sound/vibration and/or heating and/or visible light and/or IR radiation means only without any electric stimulating means and the therapeutic effect is achieved by direct heating and/or light radiation and/or sound only.

FIG. 5 shows examples of the skin contact surface of the device containing another type of device electrode assembly. On the skin-contacting surface of the device body, multiple small electrodes are attached. Some electrodes function as positive and others function as negative and they can alternate polarity during stimulation. The electrode can be small metal dot/piece/chip or other shape that protrudes from the surface. In some embodiments, a vibration producing mean (e.g. a motor) is placed underneath the skin contact surface to provide vibrational stimulation to the skin.

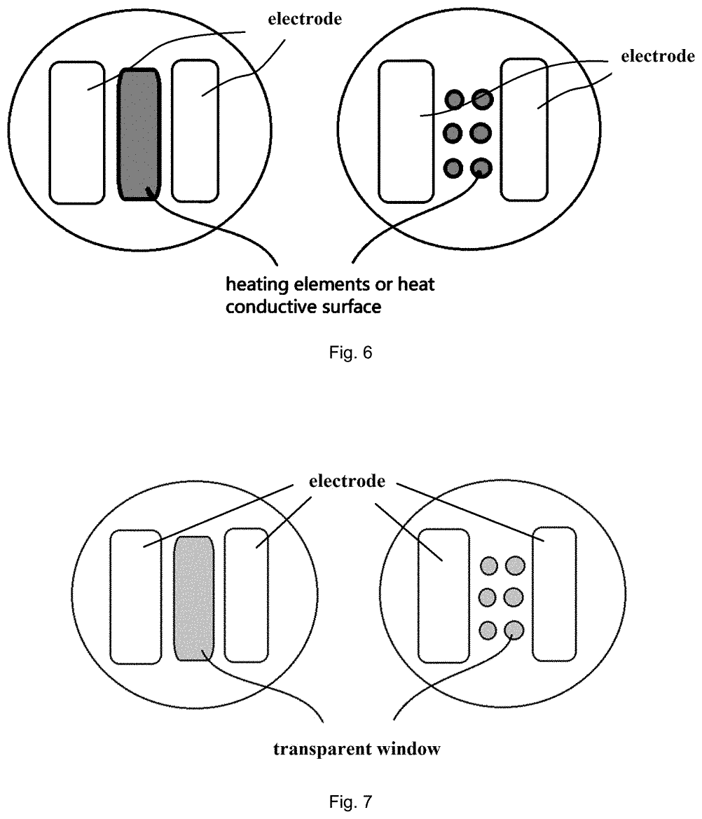

FIG. 6 shows embodiments of a device electrode assembly with heating element. On the skin-contacting surface of the device body, two electrodes are attached. Between the two electrodes is a heating element or a heat conducting area (e.g. a metal piece) with a heating element attached beneath. Alternatively, the heating element or the heat conductive area can be in an array form. Yet in another embodiments, the heat conductive area can also be the electrode.

FIG. 7 shows embodiments of device electrode assembly with light radiation element. On the skin-contacting surface of the assembly, two electrodes are attached. Between the two electrodes is a transparent window with a laser or other type of light (e.g. LED) output source incorporated beneath. Alternatively, the transparent window can be in an array form. A laser or LED light source can deliver the light to these windows with optical fibers. It can also has multiple LED embedded on the skin contacting surface.

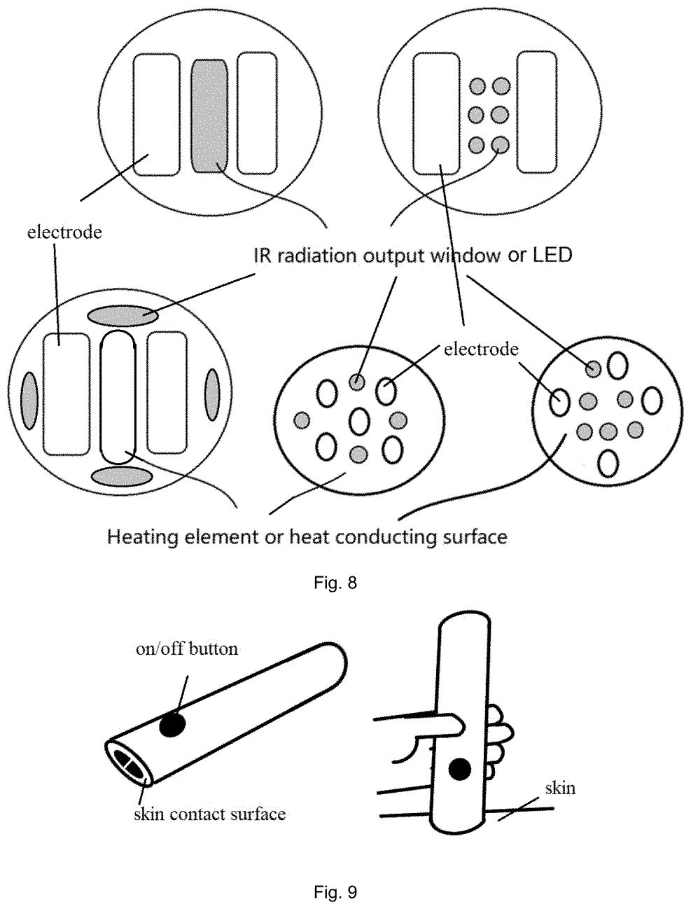

FIG. 8 shows embodiments of the device electrode assembly with light radiation element and heating element. Its skin contact surface has electrodes, light radiation window or LED, and heating elements or heating conducting surface with heating elements built underneath. In some embodiments, the heating conducting surface can be electrode surface itself.

Preferably, the device in the invention is portable and self-contained, with shape and means to be pressed against the skin area to be treated. It can be in a solid handhold shape/form of a pen, a box, a cylinder, a cube, a stamp, a cone, or other shape with a handle that can be hold with hand to place it against the skin so that one end of the device having electrodes on the surface of that end is in direct contact with the skin. The device can produce electrical pulse or heat or cold or light or IR radiation or vibration or sound or their combination and apply them to the said skin area. Exemplary suitable shapes are shown in FIGS. 9-16.

As shown in FIG. 9, an exemplary device has a shape of a pen. It has a round skin contact surface ˜2.5 cm in diameter with two half circle electrodes on it such as those shown in FIG. 4. It has an on/off button that control the on/off and intensity of electrical stimulation (voltage/current), long press will turn the device on or off and short press will change the intensity. It has a power supply (e.g. a rechargeable battery) and built in circuit can emit electric pulse that is preset in the chip within device, for example, a square wave pulse between 10-200 Hz with pulse width between (0.05˜5 ms) at 0.5˜30 mA current intensity by providing a voltage on skin surface between 10˜60V.

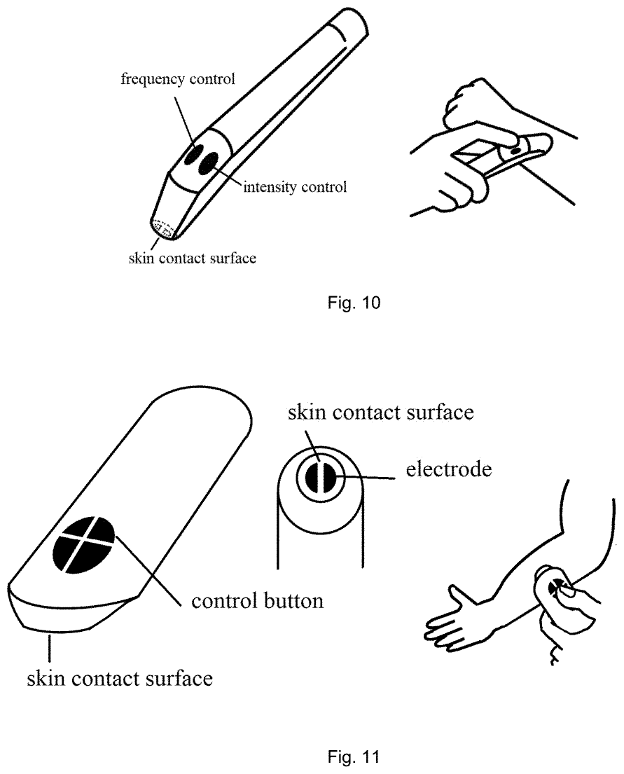

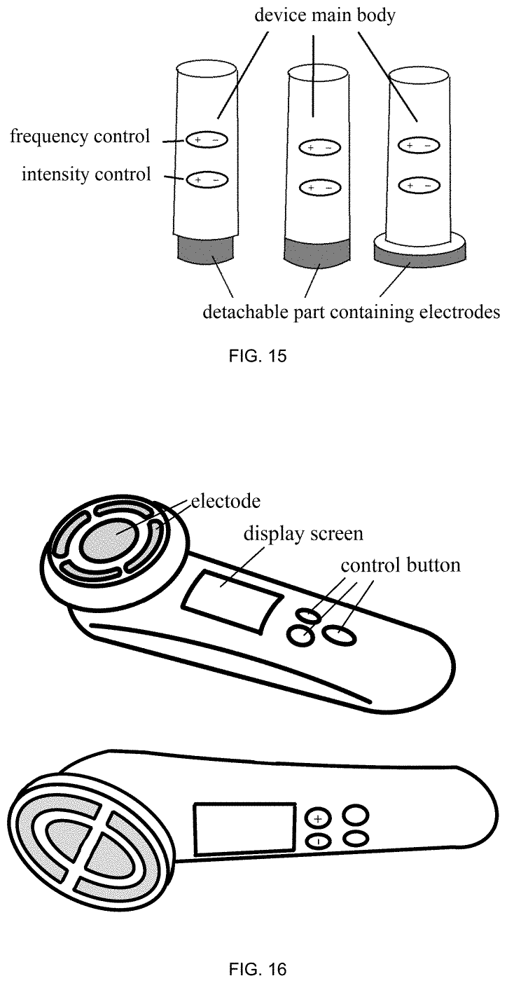

FIG. 10 shows another example of the device. It has a round skin contact surface ˜1 cm in diameter with two half circle shape electrodes on its skin contact surface as shown as the dotted line in the figure. For illustration purpose, the dotted line shows the parts not directly visible from the view angle of FIG. 10. The device has a frequency control that can adjust the frequency of the output electrical pulse between 30˜200 Hz. Repeated quick presses of the button will increase the frequency and long pressing will return the frequency to 30 Hz. Or the button can be pressed bi-directionally, where pressing one end (+end) will increase the frequency and pressing another end (−end) will decrease frequency (e.g. as those in FIG. 15). It has an intensity control that control the on/off and intensity of stimulation (voltage/current), long press will turn the device on or off and short press will change the intensity. Or the button can be pressed bi-directionally, pressing one end will increase the intensity and pressing another end will decrease intensity and can further turn the device off (e.g. as those in FIG. 15). It has a power supply (e.g. a rechargeable battery) and built in circuit can emit electric pulse controlled by the button and chip within device, for example, square wave pulse between with pulse width between (0.1˜3 ms) up to 20 mA current intensity. Optionally, the device has a built in heating means and the skin contact surface can also provide heating effect at 40-50° C. at the same time of electrical pulse stimulation. In some embodiments, the device also has a temperature control button that can adjust the surface temperature of the skin contact surface. Optionally, the device has a built in vibrational means and the skin contact surface can also provide vibrational effect to the skin at the same time of electrical pulse stimulation. In some embodiments, the device also has a vibrational control button that can adjust the vibrational intensity and/or frequency (e.g. 2˜20 Hz) of the skin contact surface.

FIG. 11 shows another example of the device, which is similar to that in FIG. 9, except its skin contact surface is ˜1 cm in diameter and has a surface temperature 39-45° C. that can also provide heating stimulation to the skin when the device is on with a built in heating circuit in the device. Alternatively, instead of the heating means the device has a built-in thermoelectric cooler such as Peltier element that can cool the skin contact surface at 3-15° C. or providing cold air flow to the target skin area. Alternatively, besides the heating means the device also has a built-in thermoelectric cooler such as a Peltier element that can cool the skin contact surface to 3-15° C. therefore providing alternating heating and cooling stimulation.

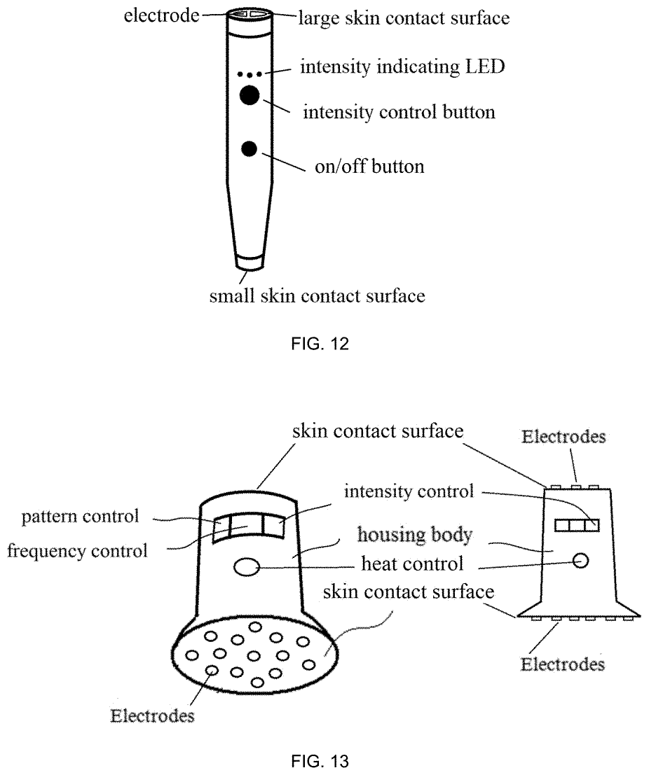

In some embodiments, the device has more than one skin contact surface having different sizes, and each skin contact surface has their own stimulation means such as electrodes, heating, cooling, light irradiation and vibrational means. One example having a pen shape is shown in FIG. 12. It has two skin contact surface containing electrodes, one is ˜1 cm²m at one end, which can be used to treat small itching area such as insect bite, another is ˜3 cm² at another end, which can be used to treat larger skin itching area.

FIG. 13 shows another example of a cone shape device that has two skin stimulation surface containing electrodes. The electrodes are small metal piece that protrude from the surface and can also deliver heat stimulation to the skin area in contact. The device housing body has built-in circuit and power source that produce electrical pulse and heating stimulation. It has a separate heat control that can adjust the heating temperature between 40° C. to 50° C. and can turn on/off the heat stimulation. The intensity control controls the intensity of electrical pulse stimulation current from 0 to 30 mA. The frequency control controls the frequency of electrical pulse stimulation from 5 Hz to 300 Hz. The pattern control electrical pulse stimulation pattern such as those describe previously, (e.g. continuous, intermittent or cycling between several different frequency, AMF, FMW or PWM). The device can further has a vibrational means (e.g. motor) built in to provide additional vibrational stimulation (e.g. 5 Hz˜50 Hz) through the skin contacting electrodes. In some embodiments, the device further has LED (e.g. 630 nm and 850 nm) on its skin contact surface to provide red light/NIR irradiation to the skin.

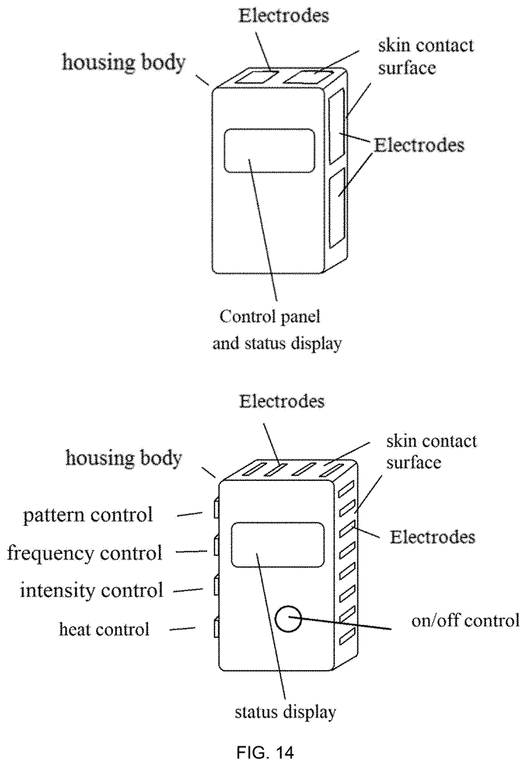

FIG. 14 top shows another example of a cube shape device that has two skin stimulation surface containing electrodes on the surface. It has two skin contact surface on the side of the cube (˜20 cm² and 40 cm² in size), one with 4 rectangular shape electrodes, which can be used to treat small itching area, and another with 8 rectangular shape electrodes, which can be used to treat a larger area with itchiness. It also has a separate on/off control to turn the device on or off and control which surface to provide stimulation. It can have a status display window, such as an LCD display. Optionally the display can also be a touch screen to allow user to control the device using touch screen therefore no additional buttons to control pattern, frequency, intensity or heat are required. Optionally the device has a built in circuit that can communicate with a remote or a cell phone (e.g. through wireless communications such as Wi-Fi or Bluetooth) and a corresponding cell phone app can control the device and program the stimulations. The electrode numbers, shape and size can also be varied as shown in FIG. 14 bottom.

Alternatively, the device can have only one skin contact surface. The surface has multiple electrodes. Only a subset of these electrodes (portion of all electrodes) will be turned on if the skin area to be treated is small and all electrodes can be turned on if the skin area to be treated is large. A control means can be incorporated within the housing body to control the stimulation area and the user can use a control button on the housing body to select the size of the skin area to be treated. Examples of the electrodes configuration can be found in FIGS. 4,5, 8 and 13. For example, a device has one skin contact surface with ˜30 electrodes on it. When the itchy area is small (e.g. a mosquito bite), only 10 electrodes in the center are active to deliver electrical pulses. When the itchy area is large (e.g. a mosquito bite), all electrodes on the surface are active to deliver electrical pulses to cover larger skin area. Similarly, the skin contact surface can have multiple heating areas and either a sub set of them or all of them can be turned on to deliver heat based on the size of the skin to be treated. In some embodiments, the heating area is electrode.

In some embodiments, as shown in FIG. 15 the device has a detachable part containing the skin contacting surface. The detachable parts having different skin contact surface area and/or different stimulation means are interchangeable to attach to the main body for different needs, e.g. to stimulate different size of itching skin area.

FIG. 16 shows examples of the device having a status display. The status display screen can be a LCD screen showing the stimulation status such as pattern, frequency, intensity and skin contact surface temperature and optionally the size of stimulation area. The device has a handle shape housing body allow user to hold it with hand, which has multiple control buttons on it to control stimulation pattern, frequency, intensity and skin contact surface temperature and the size of stimulation area. The devices have a skin contact surface around 4-6 cm in diameter with multiple electrodes (as shown in gray) embedded within. The electrode can be made of metal or other conducting material such as gel pad. One or more metal electrodes can also serve as heat conducting surface to provide heat stimulation of 40-55° C. to the skin. If gel pad electrodes are used, the non-electrode area in the skin contact surface can be made of metal to deliver heat stimulation. The electrodes can provide electrical pulse stimulation voltage between 10-100V. The device in FIG. 16 top has one round electrode and four ¼ ring shape electrodes. The device in FIG. 16 bottom has two half circle electrodes and two ½ ring shape electrodes. When the itchy area is small, the device in FIG. 16 bottom can deliver electrical pulse with the two half circle electrodes only and the two ½ ring shape electrodes will be idle without having voltage to provide electrical stimulation. When the itchy area is large, all 4 electrodes will be active to provide electrical pulse stimulation to cover larger skin area. A user can control the stimulation area size by pressing the control button on the housing body. For example, the pattern control button or dedicated stimulation area control button can be used to select the stimulation area size.

In one example, a user has skin itchiness. The user holds a device that can generates electrical pulse (e.g. those described throughout the current application such as those in FIGS. 9-15) and press the skin contact surface of the device having electrodes against the itching skin area and apply electrical pulse stimulation to that skin area. When the pulse is 5-10 Hz bidirectional square wave with pulse width of 0.5 ms at ˜2 mA current level for 6 second (intermittent wave pattern with is no pulse after 3 s continuate pulse stimulation), the itchiness is reduced by ˜50%. When concurrent heating at 45° C. is applied, the itchiness is reduced by 75%. When the pulse is 30-60 Hz bidirectional square wave with pulse width of 0.5 ms at ˜2 mA current level for 6 second (intermittent with is no pulse after 3 s continuing pulse stimulation), the itchiness is reduced by 60%. When the pulse is a 30-60 Hz bidirectional square wave with pulse width of 0.5 ms at maximal current level that the user still feels comfortable at for 6 second (intermittent with is no pulse after 3 s continues stimulation), the itchiness is reduced by 70%. When the pulse is 100-150 Hz bidirectional square wave with pulse width of 0.3 ms at half maximal current level that the user still feels comfortable at for 6 second (intermittent with 1 s no pulse after 3 s continues pulse stimulation), the itchiness is reduced by 80%. When additional vibrational stimulation (30-60 Hz) is applied, the itchiness is further reduced. When the pulse is 100-150 Hz bidirectional square wave with pulse width 0.3 ms at half maximal current level that the user still feels comfortable at for 25 second (intermittent with is no pulse after 3 s continues pulse stimulation), the itchiness is reduced by 100%. When the pulse is 100-150 Hz bidirectional square wave with pulse width 0.3 ms at half maximal current level that the user still feels comfortable at for 6 second (intermittent with is no pulse after 3 s continues pulse stimulation) and concurrent heating at 45° C., the itchiness is reduced by 100% and no itchiness feeling in the next half hour. Heating at 45° C. alone to the itchy skin area can reduce itchiness but once it is stopped itchiness comes back within a few minutes. Therefore the method of the current invention can also be used to prevent itchiness from happening. When only heating is applied, the itchiness is reduced by only ˜30% and the itchiness comes back after a few minutes. Other stimulation pulse pattern (e.g. sinusoidal wave, continued, varying amplitude) also produce similar anti-itching effect.

When the stimulation was electrical pulse of alternating 2 Hz (0.6 ms pulse width) for 3 s followed by 1 s idle and then 15 Hz (0.3 ms pulse width) for 3 s; or alternating 2 Hz (0.6 ms pulse width) for 3 s, 1 s idle and then 100 Hz (0.2 ms pulse width) for 3 s; or alternating 2 Hz (0.6 ms pulse width) for 3 s, 15 Hz (0.3 ms pulse width) for 3 s and 100 Hz (0.2 ms pulse width) for 3 s with 1 s idle between each frequency, at half maximal current level that the user still feel comfortable for 30 second, the itchiness was reduced by 100% and no itchiness feeling happened in the next hour.

It is observed that for some people, higher intensity, higher frequency and longer durations of electrical pulse stimulation can provide a better anti itching effect. The combination of low and high frequency stimulation can also provide a better effect. In most cases, 10 s to 1 min of electrical pulse stimulation is sufficient to achieve desired anti-itching effect. Longer durations of stimulation (e.g. several minutes to half hour) do not further relieve itching, but they do show higher levels of itchiness prevention effect.

The light irradiation (e.g. 670 nm and 810 nm) at the power level of the volunteer feels comfortable for ˜0.5 hour every day is applied to skin area suffering itchiness, frequency of onset of skin itchiness is reduced. Additional electrical pulse and vibrational stimulation (e.g. 10-30 min every day) in combination with said light irradiation further improve the skin conditions in term of itching prevention effect.

Therefore, the current invention discloses a method to alleviate skin itchiness as well as prevent skin itchiness comprising placing the skin contact surface of the device disclosed in the current invention on the area of skin that itches, and applying an electric pulse with one or more frequency selected from 1-500 Hz for 5 s to 1 min or until the itchiness is relieved. Additional heating and/or vibration stimulation can be applied simultaneously.

The device of the current invention can have a control means such as built-in circuit with control button/pad that can adjust the treatment time and intensity as previous described. The device can have a communication module that can communicate with an external control (command) module to receive commands of the stimulation output (e.g. stimulation type, time, frequency, on/off, power level and pulse pattern) and produce stimulation accordingly. The external control module can be a remote or computer or a cell phone with dedicated application installed. The communication can utilize Wi-Fi or Bluetooth or IR or radio signal. The device can have an on/off control to turn on or turn off the communication module. The device can have a built-in power supply such as battery or be connected to an electricity outlet.

The device can also contain a means to generate cooling stimulation (e.g. 5-15° C. to treated area) which can be applied to the treated area. The devices described in the current invention can use heating stimulation. Alternatively, cooling stimulation or both heating and cooling stimulation can be employed sequentially, such as by rotating heating and cooling stimulation. The cooling effect can be achieved by using a built-in thermoelectric cooler such as a Peltier element that can cool the skin contact surface at 5-15° C. or providing cold air flow to the target area.

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known mechanics, instruments, methods and skills. A skilled person can easily find the knowledge from textbooks such as the textbooks, scientific journal papers and other well-known reference sources.

1. A device for relieving itching in a subject in need, said device comprising:
a small housing body having a size and shape adapted to be held with hand;
a skin contact surface having two or more electrodes on said surface;
heating means that can maintain the skin contact surface temperature between 40-50 degrees;
circuit means for generating pulsed electrical stimulation signals of selected amplitude and pulse rate at a selected frequency and delivering the electrical stimulation signals to the electrodes.

2. The device of claim 1, wherein the frequency is between 5 Hz and 300 Hz.

3. The device of claim 1, wherein the circuit means can deliver the pulse with voltage between 10 and 60 volts to the electrodes.

4. The device of claim 1, further comprising a light stimulation means.

5. The device of claim 1, further comprising a vibration stimulation means.

6. A method for relieving itching in a subject in need thereof, the method comprising:
placing a handheld device having a skin contact surface against the skin area having itching, wherein the skin contact surface has multiple electrodes; and
administering a non-invasive electrical stimulus from the electrodes to the subject's itchy skin surface.

7. The method of claim 6, wherein said electrical stimulus has a pulse frequency between 5 Hz and 300 Hz.

8. The method of claim 6, further comprising administering a light stimulation from the device to the itchy skin.

9. The method of claim 6, further comprising administering a vibration stimulation from the device to the itchy skin.

10. A method for relieving itching in a subject in need thereof, the method comprising:
placing a handheld device having a skin contact surface against the skin area having itching, wherein the skin contact surface has multiple electrodes and the device has a heating means; and
administering a non-invasive electrical stimulus from the electrodes and a heat stimulus to the subject's itchy skin surface.

11. The method of claim 10, wherein said electrical stimulus has a pulse frequency between 5 Hz and 300 Hz.

12. The method of claim 10, further comprising administering a light stimulation from the device to the itchy skin.

13. The method of claim 10, further comprising administering a vibration stimulation from the device to the itchy skin.

14. The method of claim 10, where in the heat stimulus has a temperature between 40 and 50 degrees.
